(19) United States
(12) Patent Application Publication
(10) Pub. No.: US 2022/0047643 A1
(43) Pub. Date: Feb. 17, 2022

(54) FIBROBLASTS AND MICROVESICLES THEREOF FOR REDUCTION OF TOXICITY ASSOCIATED WITH CANCER IMMUNOTHERAPY

(71) Applicant: Figene, LLC, Houston, TX (US)

(72) Inventors: Pete O'Heeron, Houston, TX (US); Thomas chim, San Diego, CA (US)

(21) Appl. No.: 17/310,051

(22) PCT Filed: Jan. 17, 2020
(86) PCT No.: PCT/US2020/014018

§ 371 Date: Jul. 13, 2021

Related U.S. Application Data
(60) Provisional application No. 62/793,545, filed on Jan. 17, 2019.

(51) Int. Cl. A61K 35/33; A61K 45/06; C07K 16/24; A61K 35/17; A61P 35/00

(52) U.S. Cl. CPC A61K 35/33; A61K 45/06; A61P 35/00; A61K 35/17; C07K 16/244

(57) ABSTRACT

Embodiments of the disclosure include methods and compositions related to treatment and prevention of an excess of cytokines in an individual using fibroblasts or fibroblast-derived microvesicles. In particular embodiments, there are methods and compositions for treating and preventing toxicities in an individual that may be the result of cytokine release syndrome. In specific cases, an individual is treated for cytokine release syndrome with fibroblasts having one or more specific markers.

This application claims priority to U.S. Provisional Patent Application Ser. No. 62/793,545, filed Jan. 17, 2019, which is incorporated by reference herein in its entirety.

### TECHNICAL FIELD

Embodiments of the disclosure include at least the fields of cell biology, molecular biology, cell therapy, and medicine, including cancer.

### BACKGROUND

Malignant tumors have become one of the main diseases that threaten human health and survival. The conventional therapies of cancer, such as surgical operation, radiation therapy and chemotherapy involve external forces, which include exscinding tumors directly, killing tumor cells with radiation, or by chemotherapeutics. Chemotherapy and radiation therapy usually are unable to resolve issues such as tumor metastasis and recurrence. Moreover, these treatments always have severe toxic effects and damage normal cells. In particular, conventional radiation therapy and chemotherapy will damage the immune system, especially the NK cell and T cell-mediated immunity that plays an important role in the body's anti-tumor defense. Interestingly, in some cases the depression and fatigue associated with conventional cancer therapies may be playing a neurological role in the suppression of immunity, as evidenced by patients recovering immune scores subsequent to administration of psychological anti-depressants. Increasing attention has been paid to biological treatments that are based on immunotherapy. This approach possesses the advantages of specifically killing tumor cells while sparing non-malignant cells, thus not causing the horrific side effects commonly associated with conventional cancer therapies. Unfortunately, despite recent successes of immunotherapy, whether checkpoint inhibitors or cellular approaches such as chimeric antigen receptor (CAR)-T cell approaches, a new type of adverse effect to immunotherapy has been observed. In contrast to side effects of chemotherapy or radiation therapy, which arise because of killing of mitotically active cells, side effects of immunotherapy are related to exaggerated production of cytokines, which possesses the clinical name “cytokine release syndrome”.

Cytokine release syndrome is an adverse effect of various immunotherapies that in certain occasions is dangerous and sometimes life-threatening. One example of cytokine release syndrome is what occurs in some patients administered T cell immunotherapies such as chimeric antigen receptor (CAR)-T cells. In situations where these cells become activated, they produce a systemic inflammatory response in which there is a rapid and massive release of cytokines into the bloodstream, leading to dangerously low blood pressure, high fever and shivering, for example. In more severe cases of cytokine release syndrome, patients experience a cytokine storm (a.k.a. cytokine cascade or hypercytokinemia), in which there is a positive feedback loop between cytokines and white blood cells with highly elevated levels of cytokines. This can lead to potentially life-threatening complications including cardiac dysfunction, adult respiratory distress syndrome, neurologic toxicity, renal and/or hepatic failure, pulmonary edema and disseminated intravascular coagulation, for example.

One devastating example of cytokine storm was observed in a Phase I trial of TGN1412, an antibody that binds to the CD28 receptor on T-cells. Ninety minutes after receiving a single intravenous dose of the drug, all six volunteers had a systemic inflammatory response characterized by a rapid induction of proinflammatory cytokines and accompanied by headache, myalgias, nausea, diarrhea, erythema, vasodilatation, and hypotension. Within 12 to 16 hours after infusion, they became critically ill, with pulmonary infiltrates and lung injury, renal failure, and disseminated intravascular coagulation. Severe and unexpected depletion of lymphocytes and monocytes occurred within 24 hours after infusion. All six patients were transferred to the care at an intensive care unit at a public hospital, where they received intensive cardiopulmonary support (including dialysis), high-dose methylprednisolone, and an anti-interleukin-2 receptor antagonist antibody. Prolonged cardiovascular shock and acute respiratory distress syndrome developed in two patients, who required intensive organ support for 8 and 16 days. Despite evidence of the multiple cytokine-release syndrome, all six patients survived.

The typical treatment of cytokine release syndrome consists of corticosteroids, biological therapies, such as anti-IL6 therapies, and anti-inflammatory agents. However, steroids may affect CAR T-cells' activity and/or proliferation and put the patients in danger of sepsis and opportunistic infections. Anti-inflammatory drugs may not be effective in controlling cytokine release syndromes or cytokine storms, because the cytokine storm includes a very large number of cytokines while there is limited ability to infuse patients with anti-inflammatory drugs. Novel strategies are needed to control cytokine release syndromes, and especially cytokine storms, in order to safely utilize certain therapies, including at least CAR T-cell therapy and other immunotherapies.

### BRIEF SUMMARY

In particular embodiments, the present disclosure is directed to methods and compositions related to inhibiting or preventing toxicity of a therapy in an individual. The toxicity of any therapy may be treated or prevented, but in particular cases the methods concern inhibiting toxicity of an immunotherapy. The immunotherapy may employ as part (or all) of the immunotherapy an antibody or functional fragment thereof, including monoclonal antibodies or functional fragments thereof. In some embodiments, methods concern treatment or prevention of toxicity in an individual. Therapy toxicities of any kind may be ameliorated at least in part with methods and compositions of the present disclosure. The toxicities may be immunotherapies, radiation, drug toxicities, oxygen therapy, endocrine therapy, gene therapy, and so forth.

The present disclosure in certain embodiments comprises treatment or prevention of cytokine release syndrome or any form thereof, including systemic inflammatory response syndrome, cytokine storm, cytokine cascade or hypercytokinemia, for example.

Embodiments of the disclosure include methods of reducing cytokine levels, including deleterious levels, in an individual, including one that has cytokine release syndrome or any form thereof, including systemic inflammatory response syndrome, cytokine storm, cytokine cascade or hypercytokinemia, for example. The levels being deleterious may be determined through quantitative measurements from a sample from the individual and/or extrapolated from one or more symptoms. The levels may be monitored over time. The individual may or may not have been given a therapy that directly or indirectly resulted in cytokine release syndrome. The individual may or may not be suffering from an infectious disease or non-infectious medical condition that directly or indirectly resulted in cytokine release syndrome.

In specific embodiments of the disclosure, individuals are provided an effective amount of fibroblasts and/or fibroblast-derived microvesicles for any purpose, and in particular cases the fibroblasts are dedifferentiated fibroblasts. The fibroblasts may be dedifferentiated in any manner, but in specific embodiments they are exposed to one or more histone deacetylase inhibitors, as an example.

Embodiments of the disclosure include methods of reducing one or more inflammatory cytokines (such as TNF, for example) in an individual by providing to the individual an effective amount of fibroblasts and/or fibroblast-derived microvesicles, including fibroblasts that have been de-differentiated.

In specific embodiments, methods of the disclosure suppress or eliminate cachexia in an individual by providing to the individual an effective amount of fibroblasts and/or fibroblast-derived microvesicles, including fibroblasts that have been de-differentiated. The cachexia in the individual may be caused by any reason, including cancer, chemotherapy, chronic renal failure, HIV, and multiple sclerosis, as examples.

Particular embodiments include enhancing response in an individual to one or more therapies by administering to the individual an effective amount of fibroblasts and/or fibroblast-derived microvesicles, including fibroblasts that have been de-differentiated. The therapy may be of any kind, including a therapy that is prone to having toxicity for an individual, such as toxicity associated with excessive cytokine production in the recipient individual, for example.

Embodiments of the disclosure include methods of reducing toxicity of a therapy for an individual, comprising the step of providing an effective amount of fibroblasts and/or fibroblast-derived microvesicles to the individual with the therapy and/or before the therapy and/or after the therapy has been given to the individual. The therapy may be one or more of immunotherapy, radiation, drug toxicity, oxygen therapy, endocrine therapy, or gene therapy. The therapy may be for cancer, infectious disease, and/or autoimmunity, for example. In cases wherein the therapy is immunotherapy, the immunotherapy may comprise an antibody or functional fragment thereof. Any antibody may be employed, including, for example, a monoclonal antibody. A functional antibody fragment may comprise a scFv, as an example. In some cases, the therapy (of any kind, including immunotherapy) may comprise cells. The immunotherapy cells may be stem cells, T cells, NK cells, NK T cells, macrophages, B cells, lymphokine activated cells, tumor-infiltrating lymphocytes, and mixtures thereof; such cells may or may not be engineered to express a synthetic and/or exogenous protein, such as a receptor, a cytokine, or both, for example. In specific embodiments, the immunotherapy comprises cells expressing one or more engineered T-cell receptors (TCR) or one or more chimeric antigen receptors (CAR) or both. The TCR or CAR may target 1, 2, 3, or more cancer antigens. In cases wherein the cells express one or more CARs, the CAR may or may not comprise more than one costimulatory domain.

In specific embodiments, fibroblasts utilized in methods and compositions of the disclosure are dedifferentiated fibroblasts. Methods of the disclosure may further comprise the step of dedifferentiating the fibroblasts. In specific cases, fibroblasts are or were dedifferentiated upon exposure to a sufficient amount of one or more dedifferentiating agents. The dedifferentiating agent may be one or more histone deacetylase (HDAC) inhibitors (such as valproic acid), one or more DNMT inhibitors, hypoxia, and/or exposure to stem cells or fractions thereof, in some cases. When valproic acid is utilized, the valproic acid may be exposed to the fibroblasts at a concentration of 1-100 micrograms per milliliter for a period of 1-72 hours. In specific embodiments, the fibroblasts are derived from a tissue comprising regenerative properties. Examples of tissue include umbilical cord, placenta, or a mixture thereof.

In specific embodiments, fibroblasts of any kind that are utilized may express one or more of CD105, CD117, and/or CD34. In some cases, the fibroblasts in addition or alternatively comprise expression of rhodamine 123 efflux activity. Such markers and activity may be present in the fibroblasts before and/or after de-differentiation.

In cases wherein microvesicles are utilized, the microvesicles may comprise exosomes, apoptotic bodies, exosome-like particles, or a mixture thereof. The microvesicles may be produced from culture of de-differentiated fibroblasts using anion exchange chromatography, high performance liquid chromatography (HPLC), or both. In specific embodiments, the microvesicles express one or more markers selected from the group consisting of a) CD63; b) CD9; c) MHC I; d) CD56; and e) a combination thereof. In specific embodiments, the fibroblasts and/or fibroblast-derived microvesicles are modified to reduce macrophage activation. The fibroblasts and/or fibroblast-derived microvesicles may be comprised in polymer-augmented liposomes.

In any embodiment, the individual may be provided an effective amount of activated protein C.

Embodiments of the disclosure include methods of treating or preventing cytokine release syndrome in an individual in need of or having received a therapy, comprising the step of providing an effective amount of fibroblasts and/or fibroblast-derived microvesicles to the individual with the therapy and/or before the therapy and/or after the therapy has been given to the individual. The cytokine release syndrome may be from a therapy, an infectious disease, or a non-infectious disease, as examples. In specific cases, the non-infectious disease is graft-versus-host disease (GVHD), acute respiratory distress syndrome (ARDS), sepsis, sepsis, pancreatitis, burns, trauma, or Hemophagocytic lymphohistiocytosis. The infectious disease may be Ebola, influenza (such as avian influenza), severe acute respiratory syndrome, malaria, or smallpox. In cases of influenza, it may be Type A, Type B, or Type C influenza. In cases wherein the cytokine release syndrome is from a therapy, the therapy may be immunotherapy, such as an antibody or functional fragment thereof. In specific embodiments, the cytokine release syndrome is further defined as systemic inflammatory response syndrome, cytokine storm, cytokine cascade, or hypercytokinemia. The individual may be further provided one or more corticosteroids, one or more biological therapies, and/or one or more anti-inflammatory agents. In specific embodiments, the biological therapy comprises one or more anti-IL6 therapies, and the anti-IL6 therapy may comprise one or more anti-IL6 antibodies.

Embodiments of the disclosure include methods of reducing cytokine levels of one or more cytokines in an individual, comprising the step of providing an effective amount of fibroblasts and/or fibroblast-derived microvesicles to an individual in need of reduction of one or more cytokines. The individual may have cytokine release syndrome, which may be further defined as systemic inflammatory response syndrome, cytokine storm, cytokine cascade or hypercytokinemia.

Embodiments of the disclosure include methods of treating cachexia in an individual, comprising the step of providing to the individual an effective amount of fibroblasts and/or fibroblast-derived microvesicles to an individual.

Embodiments of the disclosure include methods of enhancing efficacy of a therapy in an individual, comprising the step of providing to the individual an effective amount of fibroblasts and/or fibroblast-derived microvesicles to an individual.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWING

For a more complete understanding of the present disclosure, reference is now made to the following descriptions taken in conjunction with the accompanying drawing, in which:

FIG. 1 shows reduction of systemic TNF-alpha by valproic acid-treated fibroblasts subsequent to PD-1 antibody. Balb/c mice were administered saline (control), antibody to PD-1 (PD-1) or antibody to PD-1 together with Balb/c fibroblasts (100,000 cells intraperitoneal) that had been cultured in valproic acid at 5 micrograms per milliliter for 24 hours. Cells and antibody were injected every second day. Serum TNF alpha was measured by ELISA.

FIG. 2 shows reduction of Lymphokine Activated Cell (LAK) Lethality by Administration of Fibroblasts. Left bar is control, second from left is LAK, second from right is LAK+ MSC, and right is LAK+ fibroblasts.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “allogeneic,” as used herein, refers to cells of the same species that differ genetically from cells of a host.

The term “autologous,” as used herein, refers to cells derived from the same individual. The term “engraft” as used herein refers to the process of stem cell incorporation into a tissue of interest in vivo through contact with existing cells of the tissue.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated fibroblasts” refers to fibroblasts treated with one or more stimuli capable of inducing one or more alterations in the cell: metabolic, immunological, growth factor-secreting, surface marker expression, and/or production of microvesicles.

As used herein, the term “activated immune cells” refers to immune cells treated with one or more stimuli capable of inducing one or more alterations in the cell: metabolic, immunological, growth factor secreting, surface marker expression, and production of microvesicles.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum. The media may be periodically changed.

The term “dedifferentiated” as used herein refers to cells possessing markers of enhanced pluripotency and plasticity subsequent to exposure to certain conditions. For example, inducible pluripotent cells are dedifferentiated forms of fibroblasts.

As used herein, the term “isolated” refers to a stem cell or population of daughter stem cells in a non-naturally occurring state outside of the body (e.g., isolated from the body or a biological sample from the body). The biological sample can include synovial fluid, blood (e.g., peripheral blood), or tissue.

The term “microvesicle” as used herein refers to a subcellular particle that is enclosed by a membrane. Microvesicles include exosomes, apoptotic bodies and cellular parts that have been shed from the cell but are membrane encapsulated

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

By a “population of cells” is meant a collection of at least ten cells. Preferably, the population consists of at least twenty cells, more preferably at least one hundred cells, and most preferably at least one thousand, or even one million cells. Because the stem cells of the present invention exhibit a capacity for self-renewal, they can be expanded in culture to produce populations of even billions of cells.

As used herein, the term “purified” as in a “purified cell” refers to a cell that has been separated from the body of a subject but remains in the presence of other cell types also obtained from the body of the subject. By “substantially purified” is meant that the desired cells are enriched by at least 20%, more preferably by at least 50%, even more preferably by at least 75%, and most preferably by at least 90% or even 95%.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “subject” or “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof.

The term “toxicity” as used herein refers to pathological alteration in health, for example, reduction of an organ's physiological activity or damage to cells or tissues.

### II. of Method Embodiments of the Disclosure

The disclosure concerns at least in part methods and compositions related to rendering therapies safe for use in mammals. In particular cases the methods and compositions inhibit toxicities from a therapy that may cause adverse effects in a recipient individual. In some cases, the therapy may include cancer immunotherapy, such as tumor immunotherapy, including inhibition of toxicity of immunotherapy using novel cellular approaches.

In one aspect, disclosed herein are methods of inhibiting or reducing the incidence of toxicity from a therapy for an individual by providing a sufficient amount of fibroblasts and/or fibroblast-derived microvesicles to the individual that has toxicity or is susceptible to having or acquiring toxicity.

Disclosed are methods, means, and compositions of matter useful for reduction of toxicity associated with immunotherapy, including anticancer immunotherapy, such as without substantially compromising therapeutic efficacy of the immunotherapy. In one embodiment, individuals undergoing immunotherapy are administered fibroblasts and/or fibroblast-derived microvesicles prior to, and/or concurrent with, and/or subsequent to treatment with one or more immunotherapies. In some embodiments, fibroblasts and/or fibroblast-derived microvesicles are generated from allogeneic cells possessing an immature phenotype that is capable of plasticity in response to cytokine environments. The plasticity may comprise the ability to inhibit systemic antigen-nonspecific inflammatory activity, while preserving tumor-specific immunotherapy activity. In specific embodiments, the tumor-specific immunotherapy activity being preserved includes T cell, NKT cell, and/or NK cell activity, as examples.

In particular embodiments, the toxicity of one or more immunotherapies is inhibited either entirely or partially upon use of fibroblasts and/or fibroblast-derived microvesicles. The intensity of a toxicity may be reduced, in some cases. In some aspects, the onset of toxicity is delayed, for example long enough for the individual to be provided a sufficient amount of one or more other therapies for the toxicity or for another reason. The methods may or may not be utilized prophylactically for an individual. In specific embodiments, an individual in need of one or more immunotherapies may be provided a sufficient amount of fibroblasts and/or fibroblast-derived microvesicles. Prior to this, during this, and/or after this, the individual is provided the one or more immunotherapies.

The fibroblasts or fibroblast-derived microvesicles may be provided to the individual for any kind of therapeutic toxicity, including toxicity for immunotherapies. In specific embodiments, the immunotherapy toxicity is cytokine release syndrome and in some cases the individual has or is susceptible to having a severe case of cytokine release syndrome including cytokine storm (that may be referred to as cytokine cascade or hypercytokinemia). The individual with cytokine release syndrome may have a reduction in blood pressure and/or a fever. In particular embodiments, the cytokine release syndrome is characterized by enhanced production of one or more cytokines. In specific embodiments, the cytokines may be selected from the group consisting of: a) TNF-alpha; b) IL-1 beta; c) IL-6; d) IL-33; e) CRP; f) IL-17; g) IL-2; h) IL12; i) IL-18; j) HMGB-1; k) interferon gamma; l) interferon alpha; and m) a combination thereof.

In certain embodiments, the individual has or is susceptible to cytokine release syndrome that is caused by administration of one or more cancer immunotherapeutics. The administration of the cancer immunotherapeutic(s) may or may not be the first time the individual has received the administration. In specific embodiments, the cancer immunotherapeutic comprises chimeric antigen receptor (CAR)-specific immune cells; such CAR-specific immune cells may be CAR-specific NK, NKT, or T cells. The CARs may be directed to any antigen, including any cancer antigen, such as any tumor antigen. The CAR may or may not be a first generation type, second generation type, or third or subsequent generation type. In specific embodiments, the CAR comprises one, two, or more costimulatory domains, including at least CD28, 4-1BB, and so forth.

In some embodiments, the individual has or is susceptible to having cytokine release syndrome that is caused by one or more infectious agents. The infectious agent(s) may or may not be selected from the group consisting of a) influenza; b) bird flu; c) severe acute respiratory syndrome (SARS); d) Epstein-Barr virus-associated hemophagocytic lymphohistiocytosis (HLH); e) bacterial sepsis; f) gram-negative sepsis; g) Dengue virus; h) malaria; i) Ebola virus; j) variola virus; k) a systemic Gram-negative bacterial infection; and l) a combination thereof.

In certain embodiments, the individual has or is susceptible to having cytokine release syndrome that is induced by one or more non-infectious causes. Non-limiting examples include non-infectious causes selected from the group consisting of: a) hemophagocytic lymphohistiocytosis (HLH); b) sporadic HLH, macrophage activation syndrome (MAS), c) chronic arthritis; d) systemic Juvenile Idiopathic Arthritis (sJIA); e) Still's Disease; f) a Cryopyrin-associated Periodic Syndrome (CAPS); g) Familial Cold Auto-inflammatory Syndrome (FCAS); h) Familial Cold Urticaria (FCU); i) Muckle-Well Syndrome (MWS); j) Chronic Infantile Neurological Cutaneous and Articular (CINCA) Syndrome; k) a cryopyrinopathy comprising inherited or de novo gain of function mutations in the NLRP3 gene; l) a hereditary auto-inflammatory disorder; m) acute pancreatitis; n) severe burn injury; o) acute radiation syndrome; p) trauma; q) acute respiratory distress syndrome; r) systemic inflammatory response syndrome; and s) a combination thereof.

In particular embodiments, the individual has or may develop cytokine release syndrome or a cytokine storm. The individual may or may not be undergoing (and/or has undergone and/or will undergo) one or more immune modulatory cancer therapies, such as therapy comprising CAR-expressing immune cells (including at least NK, NKT, or T-cells). In particular embodiments, the method comprises administering one or more compositions comprising fibroblasts and/or fibroblast-derived microvesicles, wherein the administration inhibits or reduces or delays or prevents the incidence of the cytokine release syndrome or cytokine storm in the individual. Administration of compositions comprising fibroblast and/or fibroblast-derived microvesicles may occur prior to, concurrent with, and/or following the immune modulatory therapy, such as CAR T-cell therapy. In some embodiments of the disclosure, fibroblasts and/or fibroblast-derived microvesicles are administered concurrently with an immunotherapy to reduce cytokine release signal, including while enhancing T cell memory responses, as described in United States Patent Application Publication US 2002/0086027, which is incorporated by reference herein in its entirety.

Treatment with fibroblasts and/or fibroblast-derived microvesicles may be utilized within the context of the current disclosure to treat any kind of toxicity associated with a therapy, including cytokine storm, a severe manifestation of cytokine release syndrome. Cytokine storms are also a concern following infectious or non-infectious stimuli. In a cytokine storm, numerous pro-inflammatory cytokines, such as interleukin-1 (IL-1), IL-6, IL-8, γ-interferon (γ-IFN), macrophage inflammatory protein-1α (MIP-1α), tumor necrosis factor-alpha (TNFα), or a combination thereof, are released, resulting in hypotension, hemorrhage, and, ultimately, multi-organ failure. The relatively high death rate in young people, with presumably healthy immune systems, in the 1918 H1N1 influenza pandemic and the more recent bird flu H5N1 infection are attributed to cytokine storms. This syndrome has been also known to occur in advanced or terminal cases of severe acute respiratory syndrome (SARS), Epstein-Barr virus-associated hemophagocytic lymphohistiocytosis, gram-negative sepsis, malaria and numerous other infectious diseases, including Ebola infection. Cytokine storm may also stem from non-infectious causes, such as acute pancreatitis, severe burns or trauma, or acute respiratory distress syndrome. Novel strategies are therefore needed to control cytokine release syndrome, and especially cytokine storms.

In one aspect of the disclosure, fibroblasts and/or fibroblast-derived microvesicles are used to reduce macrophage activation, which plays an important role in immunological cascades associated with cytokine release syndrome. Accordingly, in some embodiments, the disclosure provides for the selective targeting of fibroblasts and/or fibroblast-derived microvesicles to macrophages and/or other cells of the reticuloendothelial system. In one embodiment, fibroblasts are administered in the form of liposomal preparations that possess enhanced ability to deliver fibroblast to the reticuloendothelial system. For example, in one embodiment, the use of polymer-augmented liposomes is provided. Examples of polymers useful for generation of polymer-augmented liposomes include: poly-1-lysine, polyamidoamine dendrimers, and polyetheleneimine. Descriptions of polymer-augmented liposomes are described in the literature.

In particular embodiments, the disclosure provides prevention or amelioration of at least one symptom of any form of cytokine release syndrome, including at least Systemic Inflammatory Response Syndrome (SIRS), by administration of fibroblasts and/or fibroblast-derived microvesicles. In the context of the disclosure, SIRS is a term characterizing an inflammatory syndrome caused by infectious, traumatic, or other causes in which patients exhibit at least two of the following criteria: 1) Body temperature less than 36° C. or greater than 38° C.; 2) Heart rate greater than 90 beats per minute; 3) Tachypnea, with greater than 20 breaths per minute; or, an arterial partial pressure of carbon dioxide less than 4.3 kPa (32 mmHg: 4) White blood cell count less than 4000 cells/mm³ (4×10⁹ cells/L) or greater than 12,000 cells/mm³ (12×10⁹ cells/L); or the presence of greater than 10% immature neutrophils (band forms). SIRS is different than sepsis in that in sepsis an active infection is found. These patients may progress to acute kidney failure; lung failure, shock, and/or multiple organ dysfunction syndrome. The term “septic shock” refers to conditions in which the patient has a systolic blood pressure of less than 90 mmHg despite sufficient fluid resuscitation and administration of vasopressors/inotropes.

Predominant events that may occur with cytokine release syndrome and such as in the progression to SIRS and subsequently to multiple organ failure are inhibited by fibroblasts and/or fibroblast-derived microvesicles within the context of the current disclosure. Such events include the following, in certain cases: a) systemic activation of inflammatory responses; b) endothelial activation and initiation of the clotting cascade, associated with consumption of anticoagulants and fibrinolytic factors; c) complement activation; and d) organ failure and death. These pathological events appear to be related to each other, for example, it is known that complement activation stimulates the pro-coagulant state. In the cancer patient, SIRS may be initiated by several factors. Numerous patients receive immune suppressive chemo/radiotherapies that promote opportunistic infections. Additionally, given that approximately 40-70% of patients are cachectic, the low grade inflammation causing the cachexia could augment effects of additional bacterial/injury-induced inflammatory cascades. Finally, tumors themselves, and through interaction with host factors, have been demonstrated to generate systemically-acting inflammatory mediators such as IL-1, IL-6, and TNF-alpha that may predispose to SIRS. Without being bound to theory, one mechanism of action of fibroblasts may be reduction of inflammatory cytokines, such as TNF, in order to suppress cachexia and enhance possibility of response to therapy.

In one embodiment, fibroblasts are combined with Xigris (activated protein C (APC)) for suppression of cytokine release syndrome. It is known that Xigris exerts its effects by activating endothelial cell-protecting mechanisms mediating protection against apoptosis, stimulation of barrier function through the angiopoietin/Tie-2 axis, and by reducing local clotting. The basis of approval for Xigris has been questioned by some and, additionally, it is often counter-indicated in oncology-associated sepsis (especially leukemias where bleeding is an issue of great concern). In fact, in the Phase III trials of Xigris, hematopoietic transplant patients were excluded.

One of the main causes of death related to SIRS is dysfunction of the microcirculatory system, which in the most advanced stages is manifested as disseminated intravascular coagulation (DIC). Inflammatory mediators associated with SIRS, whether endotoxin or injury-related signals such as TLR agonists or HMGB-1, are all capable of activating endothelium systemically. Under physiological conditions, the endothelial response to such mediators is local and provides a useful mechanism for sequestering an infection and allowing immune attack. In SIRS, the fact that the response is systemic causes disastrous consequences including organ failure. The characteristics of this endothelial response include: a) upregulation of tissue factor (TF) and suppression of endothelial inhibitors of coagulation such as protein C and the antithrombin system causing a pro-coagulant state; b) increased expression of adhesion molecules which elicit, in turn, neutrophil extravasation; c) decreased fibrinolytic capacity; and d) increased vascular permeability/non-responsiveness to vaso-dilators and vasoconstrictors. Reviews of molecular signals associated with SIRS-induced endothelial dysfunction have been published and one of the key factors implicated has been NF-kB. Nuclear translocation of NF-kB is associated with endothelial upregulation of pro-thrombotic molecules and suppressed fibrinolysis. In an elegant study, Song et al. inhibited NF-kB selectively in the endothelium by creation of transgenic mice transgenic expressing exogenous i-kappa B (the NF-kB inhibitor) specifically in the vasculature. In contrast to wild-type animals, the endothelial cells of these transgenic mice experienced substantially reduced expression of tissue factor while retaining expression of endothelial protein C receptor and thrombomodulin subsequent to endotoxin challenge. Furthermore, expression of NF-B was associated with generation of TNF-alpha as a result of TACE activity.

Immunotherapies

In some embodiments, toxicity of immunotherapies is addressed by methods and compositions encompassed herein. The toxicity may be associated with cell therapy of any kind, including stem cells, T cells, NK cells, NK T cells, macrophages, B cells, lymphokine activated cells, tumor-infiltrating lymphocytes, and mixtures thereof; such cells may or may not be engineered to express a synthetic and/or exogenous protein, such as a receptor, a cytokine, or both, for example. In specific embodiments, any immunotherapy comprising cell therapy may comprise cells with engineered antigen receptors, such as CARs, TCRs, chimeric cytokine receptors, and so forth.

In the art, treatment of toxicity associated with CAR-T cell administration by pentoxyfilline or various formulations may be applied to a variety of CAR-expressing cells. In one embodiment, the CAR-expressing cell is comprised of a CAR that binds to an epitope of an antigen via an antibody or an antibody fragment that is directed to the antigen. In another embodiment, the antibody is a monoclonal antibody. In another embodiment, the antibody is a polyclonal antibody. In another embodiment, the antibody fragment is a single-chain variable fragment (scFv).

In particular embodiment, the CAR-expressing cells of the compositions as disclosed herein bind to one or more tumor associated antigens (TAAs). A cell may comprise multiple CAR molecules or one CAR molecule. A CAR molecule may target one antigen or may target two or more antigens.

Examples of CAR-expressing cells herein may be illustrated as being T cells, although any immune cells may be modified with one or more CARs, including at least NK cells or NKT cells. In an embodiment, the tumor associated antigen is: Mucin 1, cell surface associated (MUC1) or polymorphic epithelial mucin (PEM), Arginine-rich, mutated in early stage tumors (Armet), Heat Shock Protein 60 (HSP60), calnexin (CANX), methylenetetrahydrofolate dehydrogenase (NADP+ dependent) 2, methenyltetrahydrofolate cyclohydrolase (MTHFD2), fibroblast activation protein (FAP), matrix metallopeptidase (MMP6), B Melanoma Antigen-1 (BAGE-1), aberrant transcript of N-acetyl glucosaminyl transferase V (GnTV), Q5H943, Carcinoembryonic antigen (CEA), Pmel, Kallikrein-4, Mammaglobin-1, MART-1, GPR143-OA1, prostate specific antigen (PSA), TRP1, Tyrosinase, FGP-5, NEU proto-oncogene, Aft, MMP-2, prostate specific membrane antigen (PSMA), Telomerase-associated protein-2, Prostatic acid phosphatase (PAP), Uroplakin II or Proteinase 3. Other types of CAR-T cells are known and include ones possessing a CAR that binds to CD19 or CD20 to target B cells in the case where one would like to destroy B cells as in leukemia. In another embodiment, the CAR binds to ROR1, CD22, or GD2. In another embodiment, the CAR binds to NY-ESO-1. In another embodiment, the CAR binds to MAGE family proteins. In another embodiment, the CAR binds to mesothelin. In another embodiment, the CAR binds to c-erbB2. In another embodiment, the CAR binds to mutational antigens that are tumor specific, such as BRAFV600E mutations and BCR-ABL translocations. In another embodiment, the CAR binds to viral antigens which are tumor-specific, such as EBV in HD, HPV in cervical cancer, and polyomavirus in Merkel cancer. In another embodiment, the CAR T-cell binds to Her2/neu. In another embodiment, the CAR T-cell binds to EGFRvIII.

In one embodiment, the chimeric antigen receptor (CAR) T-cell binds the CD19 antigen. In another embodiment, the CAR binds the CD22 antigen. In another embodiment, the CAR binds to alpha folate receptor. In another embodiment, the CAR binds to CAIX. In another embodiment, the CAR binds to CD20. In another embodiment, the CAR binds to CD23. In another embodiment, the CAR binds to CD24. In another embodiment, the CAR binds to CD30. In another embodiment, the CAR binds to CD33. In another embodiment, the CAR binds to CD38. In another embodiment, the CAR binds to CD44v6. In another embodiment, the CAR binds to CD44v7/8. In another embodiment, the CAR binds to CD123. In another embodiment, the CAR binds to CD171. In another embodiment, the CAR binds to carcinoembryonic antigen (CEA). In another embodiment, the CAR binds to EGFRvIII. In another embodiment, the CAR binds to EGP-2. In another embodiment, the CAR binds to EGP-40. In another embodiment, the CAR binds to EphA2. In another embodiment, the CAR binds to Erb-B2. In another embodiment, the CAR binds to Erb-B 2, 3, 4. In another embodiment, the CAR binds to Erb-B3/4. In another embodiment, the CAR binds to FBP. In another embodiment, the CAR binds to fetal acetylcholine receptor. In another embodiment, the CAR binds to G.sub.D2. In another embodiment, the CAR binds to G.sub.D3. In another embodiment, the CAR binds to HER2. In another embodiment, the CAR binds to HMW-MAA. In another embodiment, the CAR binds to IL-11Ralpha. In another embodiment, the CAR binds to IL-13Ralpha1. In another embodiment, the CAR binds to KDR. In another embodiment, the CAR binds to kappa-light chain. In another embodiment, the CAR binds to Lewis Y. In another embodiment, the CAR binds to L1-cell adhesion molecule. In another embodiment, the CAR binds to MAGE-A1. In another embodiment, the CAR binds to mesothelin. In another embodiment, the CAR binds to CMV infected cells. In another embodiment, the CAR binds to MUC1. In another embodiment, the CAR binds to MUC16. In another embodiment, the CAR binds to NKG2D ligands. In another embodiment, the CAR binds to NY-ESO-1 (amino acids 157-165). In another embodiment, the CAR binds to oncofetal antigen (h5T4). In another embodiment, the CAR binds to PSCA. In another embodiment, the CAR binds to PSMA. In another embodiment, the CAR binds to ROR1. In another embodiment, the CAR binds to TAG-72. In another embodiment, the CAR binds to VEGF-R2 or other VEGF receptors. In another embodiment, the CAR binds to B7-H6. In another embodiment, the CAR binds to CA9. In another embodiment, the CAR binds to .alpha..sub.v.beta..sub.6 integrin. In another embodiment, the CAR binds to 8H9. In another embodiment, the CAR binds to NCAM. In another embodiment, the CAR binds to fetal acetylcholine receptor. In another embodiment, the chimeric antigen receptor (CAR) T-cell targets the CD19 antigen, and has a therapeutic effect on subjects with B-cell malignancies, ALL, Follicular lymphoma, CLL, and Lymphoma. In another embodiment, the CAR T-cell targets the CD22 antigen, and has a therapeutic effect on subjects with B-cell malignancies. In another embodiment, the CAR T-cell targets alpha folate receptor or folate receptor alpha, and has a therapeutic effect on subjects with ovarian cancer or epithelial cancer. In another embodiment, the CAR T-cell targets CAIX or G250/CAIX, and has a therapeutic effect on subjects with renal cell carcinoma. In another embodiment, the CAR T-cell targets CD20, and has a therapeutic effect on subjects with Lymphomas, B-cell malignancies, B-cell lymphomas, Mantle cell lymphoma and, indolent B-cell lymphomas. In another embodiment, the CAR T-cell targets CD23, and has a therapeutic effect on subjects with CLL. In another embodiment, the CAR T-cell targets CD24, and has a therapeutic effect on subjects with pancreatic adenocarcinoma. In another embodiment, the CAR T-cell targets CD30, and has a therapeutic effect on subjects with Lymphomas or Hodgkin lymphoma. In another embodiment, the CAR T-cell targets CD33, and has a therapeutic effect on subjects with AML. In another embodiment, the CAR T-cell targets CD38, and has a therapeutic effect on subjects with Non-Hodgkin lymphoma. In another embodiment, the CAR T-cell targets CD44v6, and has a therapeutic effect on subjects with several malignancies. In another embodiment, the CAR T-cell targets CD44v7/8, and has a therapeutic effect on subjects with cervical carcinoma. In another embodiment, the CAR T-cell targets CD123, and has a therapeutic effect on subjects with myeloid malignancies. In another embodiment, the CAR T-cell targets CEA, and has a therapeutic effect on subjects with colorectal cancer. In another embodiment, the CAR T-cell targets EGFRvII, and has a therapeutic effect on subjects with Glioblastoma. In another embodiment, the CAR T-cell targets EGP-2, and has a therapeutic effect on subjects with multiple malignancies. In another embodiment, the CAR T-cell targets EGP-40, and has a therapeutic effect on subjects with colorectal cancer. In another embodiment, the CAR T-cell targets EphA2, and has a therapeutic effect on subjects with glioblastoma. In another embodiment, the CAR T-cell targets Erb-B2 or ErbB3/4, and has a therapeutic effect on subjects with Breast cancer and others, prostate cancer, colon cancer, various tumors. In another embodiment, the CAR T-cell targets Erb-B 2, 3, 4, and has a therapeutic effect on subjects with Breast cancer and others. In another embodiment, the CAR T-cell targets FBP, and has a therapeutic effect on subjects with ovarian cancer. In another embodiment, the CAR T-cell targets fetal acetylcholine receptor, and has a therapeutic effect on subjects with Rhabdomyosarcoma. In another embodiment, the CAR T-cell targets G.sub.D2, and has a therapeutic effect on subjects with neuroblastoma, melanoma, or Ewing's sarcoma. In another embodiment, the CAR T-cell targets GD3, and has a therapeutic effect on subjects with melanoma. In another embodiment, the CAR T-cell targets HER2, and has a therapeutic effect on subjects with medulloblastoma, pancreatic adenocarcinoma, glioblastoma, osteosarcoma, or ovarian cancer. In another embodiment, the CAR T-cell targets HMW-MAA, and has a therapeutic effect on subjects with melanoma. In another embodiment, the CAR T-cell targets IL-11Ralpha, and has a therapeutic effect on subjects with osteosarcoma. In another embodiment, the CAR T-cell targets IL-13Ralpha1, and has a therapeutic effect on subjects with Glioma, Glioblastoma, or medulloblastoma. In another embodiment, the CAR T-cell targets IL-13 receptor alpha2, and has a therapeutic effect on subjects with several malignancies. In another embodiment, the CAR T-cell targets KDR, and has a therapeutic effect on subjects with tumors by targeting tumor neovasculature. In another embodiment, the CAR T-cell targets kappa-light chain, and has a therapeutic effect on subjects with B-cell malignancies (B-NHL, CLL). In another embodiment, the CAR T-cell targets Lewis Y, and has a therapeutic effect on subjects with various carcinomas or epithelial-derived tumors. In another embodiment, the CAR T-cell targets L1-cell adhesion molecule, and has a therapeutic effect on subjects with Neuroblastoma. In another embodiment, the CAR T-cell targets MAGE-A1 or HLA-A1 MAGE A1, and has a therapeutic effect on subjects with Melanoma. In another embodiment, the CAR T-cell targets mesothelin, and has a therapeutic effect on subjects with Mesothelioma. In another embodiment, the CAR T-cell targets CMV infected cells, and has a therapeutic effect on subjects with CMV. In another embodiment, the CAR T-cell targets MUC1, and has a therapeutic effect on subjects with breast or ovarian cancer. In another embodiment, the CAR T-cell targets MUC16, and has a therapeutic effect on subjects with ovarian cancer. In another embodiment, the CAR T-cell targets NKG2D ligands, and has a therapeutic effect on subjects with myeloma, ovarian, and other tumors. In another embodiment, the CAR T-cell targets NY-ESO-1 (157-165) or HLA-A2 NY-ESO-1, and has a therapeutic effect on subjects with multiple myeloma. In another embodiment, the CAR T-cell targets oncofetal antigen (h5T4), and has a therapeutic effect on subjects with various tumors. In another embodiment, the CAR T-cell targets PSCA, and has a therapeutic effect on subjects with prostate carcinoma. In another embodiment, the CAR T-cell targets PSMA, and has a therapeutic effect on subjects with prostate cancer/tumor vasculature. In another embodiment, the CAR T-cell targets ROR1, and has a therapeutic effect on subjects with B-CLL and mantle cell lymphoma. In another embodiment, the CAR T-cell targets TAG-72, and has a therapeutic effect on subjects with adenocarcinomas or gastrointestinal cancers. In another embodiment, the CAR T-cell targets VEGF-R2 or other VEGF receptors, and has a therapeutic effect on subjects with tumors by targeting tumor neovasculature. In another embodiment, the CAR T-cell targets CA9, and has a therapeutic effect on subjects with renal cell carcinoma. In another embodiment, the CAR T-cell targets CD171, and has a therapeutic effect on subjects with renal neuroblastoma. In another embodiment, the CAR T-cell targets NCAM, and has a therapeutic effect on subjects with neuroblastoma. In another embodiment, the CAR T-cell targets fetal acetylcholine receptor, and has a therapeutic effect on subjects with rhabdomyosarcoma. In one embodiment the CAR binds to an angiogenic factor, thereby targeting tumor vasculature. In one embodiment, the angiogenic factor is VEGFR2. in another embodiment, the angiogenic factor is endoglin. In another embodiment, an angiogenic factor disclosed herein is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods disclosed herein is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-.beta.), endoglin and TGF-beta receptors; monocyte chemotactic protein-1 (MCP-1); Integrins .alpha.V.beta.3, .alpha.V.beta.5 and .alpha.5.beta.1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods disclosed herein is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGFbeta co-receptor. In another embodiment, the CAR T-cells bind to an antigen associated with an infectious agent. In one embodiment, the infectious agent is Mycobacterium tuberculosis. In one embodiment, said Mycobacterium tuberculosis associated antigen is: Antigen 85B, Lipoprotein IpqH, ATP dependent helicase putative, uncharacterized protein Rv0476/MT04941 precursor or uncharacterized protein Rv1334/MT1376 precursor. In another embodiment, the CAR binds to an antibody. In one embodiment, the CAR T-cell is an “antibody-coupled T-cell receptor” (ACTR). According to this embodiment, the CAR T-cell is a universal CAR T-cell. In another embodiment, the CAR T-cell having an antibody receptor is administered before, after, or at the same time as the antibody is administered and then binds to the antibody, bringing the T-cell in close proximity to the tumor or cancer. In another embodiment, the antibody is directed against a tumor cell antigen. In another embodiment, the antibody is directed against CD20. In another embodiment, the antibody is rituximab.

In another embodiment, fibroblast or formulations thereof are used to reduce cytokine release syndrome associated with administration of a therapeutic antibody. The antibody may be of any type and includes fragments such as Fab′, Fab, F(ab′)2, single domain antibodies (DABs), Fv, scFv (single chain Fv), and the like. The antibody may be polyclonal or monoclonal. In one embodiment the antibody is Trastuzumab (Herceptin; Genentech): humanized IgG1, which is directed against ERBB2. In another embodiment, the antibody is Bevacizumab (Avastin; Genentech/Roche): humanized IgG1, which is directed against VEGF. In another embodiment, the antibody is Cetuximab (Erbitux; Bristol-Myers Squibb): chimeric human-murine IgG1, which is directed against EGFR. In another embodiment, the antibody is Panitumumab (Vectibix; Amgen): human IgG2, which is directed against EGFR. In another embodiment, the antibody is Ipilimumab (Yervoy; Bristol-Myers Squibb): IgG1, which is directed against CTLA4. In another embodiment, the antibody is Alemtuzumab (Campath; Genzyme): humanized IgG1, which is directed against CD52. In another embodiment, the antibody is Ofatumumab (Arzerra; Genmab): human IgG1, which is directed against CD20. In another embodiment, the antibody is Gemtuzumab ozogamicin (Mylotarg; Wyeth): humanized IgG4, which is directed against CD33. In another embodiment, the antibody is Brentuximab vedotin (Adcetris; Seattle Genetics): chimeric IgG1, which is directed against CD30. In another embodiment, the antibody is 90Y-labelled ibritumomab tiuxetan (Zevalin; IDEC Pharmaceuticals): murine IgG1, which is directed against CD20. In another embodiment, the antibody is ¹³¹I-labelled tositumomab (Bexxar; GlaxoSmithKline): murine IgG2, which is directed against CD20. In another embodiment, the antibody is Ramucirumab, which is directed against vascular endothelial growth factor receptor-2 (VEGFR-2). In another embodiment, the antibody is ramucirumab (Cyramza Injection, Eli Lilly and Company), blinatumomab (BLINCYTO, Amgen Inc.), pembrolizumab (KEYTRUDA, Merck Sharp & Dohme Corp.), obinutuzumab (GAZYVA, Genentech, Inc.; previously known as GA101), pertuzumab injection (PERJETA, Genentech, Inc.), or denosumab (Xgeva, Amgen Inc.). In another embodiment, the antibody is Basiliximab (Simulect; Novartis). In another embodiment, the antibody is Daclizumab (Zenapax; Roche). In another embodiment, the antibody to which the CAR T-cell is coupled is directed to a tumor or cancer antigen or a portion thereof, that is described herein and/or that is known in the art. In another embodiment, the antibody to which the CAR T-cell is couples is directed to a tumor-associated antigen. In another embodiment, the antibody to which the CAR T-cell is coupled is directed to a tumor-associated antigen or a portion thereof that is an angiogenic factor.

Embodiments of the disclosure include preparations of an immunotherapy to be used with the fibroblasts and/or fibroblast-derived microvesicles. That is, in some cases the party that is making and/or using the immunotherapy is also the party that is making and/or using the fibroblasts and/or fibroblast-derived microvesicles. In some cases, however, an immunotherapy is obtained from a party that does not prepare and/or use the fibroblasts and/or fibroblast-derived microvesicles. In some methods, an individual is recognized as needing an immunotherapy based on having one or more symptoms of a medical condition for which the immunotherapy would be effective and/or based on the individual having a formal diagnosis of the medical condition. The immunotherapy and the fibroblast and/or fibroblast-derived microvesicle therapy are prepared and provided to the individual concomitantly and/or at different times. As an example, the fibroblast and/or fibroblast-derived microvesicle therapy may be provided to the individual for a sufficient amount of time before onset of the immunotherapy. As another example, the immunotherapy may be provided to the individual for a sufficient amount of time before onset of the fibroblast and/or fibroblast-derived microvesicle therapy. In any case, the route of delivery of the immunotherapy may or may not be the same route of delivery of the fibroblast and/or fibroblast-derived microvesicle therapy. In some cases, the immunotherapy and/or the fibroblast and/or fibroblast-derived microvesicle therapy are provided to the individual once or more than once. In cases wherein the immunotherapy is provided more than once, the subsequent administrations of the immunotherapy may comprise different types and/or amounts of the immunotherapy. For example, the immunotherapy may comprise a different antibody or, in the case of CAR therapy, the CAR may be directed against a different antigen(s) as the first CAR therapy. In cases wherein the fibroblast and/or fibroblast-derived microvesicle therapy is provided more than once, the subsequent administrations of the fibroblast and/or fibroblast-derived microvesicle therapy may comprise different fibroblasts and/or fibroblast-derived microvesicles. For example, the may comprise one or more different markers, may be derived from different tissue sources, may comprise different microvesicles, may be derived from different individuals, a combination thereof, and so forth.

### III. Fibroblasts and Production or Manipulation Thereof

In specific embodiments, the fibroblasts are manipulated prior to delivery to an individual for any purpose. For example, the fibroblasts may be pretreated with one or more agents capable of inducing dedifferentiation of the fibroblasts. For example, prior to use in the therapeutic methods of the disclosure, the fibroblasts may be treated with one or more histone deacetylase inhibitors (such as valproic acid); one or more DNA methyl transferase inhibitors; exposure to stem cells; hypoxia; combinations thereof; and so forth. Particular treatments for the fibroblasts may be utilized to induce dedifferentiation, such as being treated with a concentration of 1-100 (or 1-75 or 1-50 or 1-25, or 10-100 or 10-75 or 10-50 or 10-25 or 25-100 or 50-100 or 75-100 or 25-100 or 25-75 or 25-50 or 50-100 or 50-75 or 75-100, for example) micrograms per milliliter of the exemplary histone deacetylase inhibitor valproic acid for a certain period of time (as an example, between 1-72 (or 1-48 or 1-36 or 1-24 or 1-18 or 1-12 or 1-6 or 6-72 or 6-48 or 6-36 or 6-24 or 6-18 or 6-12 or 12-72 or12-48 or 12-36 or 12-24 or 12-18 or 18-72 or 18-48 or 18-36 or 18-24 or 24-72 or 24-48 or 24-36 or 36-72 or 36-48) hours.

In particular embodiments, fibroblasts may be derived from certain tissues instead of any tissue that comprises fibroblasts. In specific embodiments, the fibroblasts are derived from one or more tissues possessing regenerative properties. As an example, the fibroblasts are derived from placental tissue or umbilical cord tissue. In any method, the fibroblasts may be freshly extracted prior to manipulation for methods of the disclosure.

In some cases, the fibroblasts cells, whether or not they are derived from particular tissues, may be selected for comprising one or more specific markers. Examples include fibroblast cells selected for expression of CD105, CD117, and/or CD34. In some cases, the fibroblasts alternatively or addition to expressing CD105, CD117, and/or CD34 are selected for expression of rhodamine 123 efflux activity.

In one embodiment, allogeneic fibroblasts are administered to an individual in a non-manipulated manner (for example, without prior exposure to one or more particular agents, such as interferon gamma) but selected from sources naturally characterized by immune modulatory activity, such as placental fibroblasts or adipose tissue-associated fibroblasts, for example. In other embodiments of the disclosure, any fibroblasts are cultured under conditions capable of inducing retro-differentiation so as to endow an immature phenotype for the fibroblasts, wherein the immature phenotype correlates with enhanced anti-inflammatory and/or immune modulatory potential. For example, fibroblasts may be cultured in the presence of one or more histone deacetylase inhibitors, such as valproic acid (Moon et al., 2008; Huang et al., 2011). In addition to HDAC inhibitors, other means of inducing dedifferentiation of the fibroblasts may also be utilized in the context of the current disclosure, such as 8-Br-cAMP (Wang et al., 2011); M-CSF treatment (Li et al., 2016); exposure to reveresine (Li et al., 2016); and/or exposure to stem cell extracts (Xiong et al., 2014). Characterization of fibroblast dedifferentiation can be performed by assessment of extracellular markers, such as, such as CXCR4, VEGFR-2, CD34, and/or CD133, as well as intracellular markers such as SOX-2, NANOG, and/or OCT-4.

The fibroblasts utilized in methods and compositions, or generation of particular methods and compositions, may be fibroblasts derived from tissues adjacent to or among cells selected from the group consisting of: salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, interstitial kidney cells, or a mixture thereof.

For use within the context of the disclosure, fibroblasts and/or fibroblast-derived microvesicles may be formulated by including one or more pharmaceutically acceptable carriers in addition to an active ingredient for administration. Examples of carriers, excipients or diluents which may be included in the anticancer adjuvant of the present invention include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, or mineral oil, but are not limited thereto.

For use as an oral or parenteral formulation, fibroblast may be administered as a capsule, a tablet, a coated tablet, a slow-releasing tablet, granules, powder, syrup, a suspension, an emulsion, sap, an aerosol, and a suppository, and the parenteral preparation may be a sterilized aqueous solution, a non-aqueous solvent, a suspension, an emulsion, and a lyophilized preparation. The parenteral preparations may be administered in a typical method through an intravenous, intra-arterial, intraperitoneal, intramuscular, intrasternal, topical, rectal, or intradermal route.

Fibroblast for oral administration may be formulated with pharmaceutically acceptable carriers which typically would include a diluent, a preservative, a binder, a lubricant, a disintegrant, a swelling agent, a filler, a stabilizer, and a combination thereof, but are not limited thereto. Carriers may also include all the components of a coating composition which may include a plasticizer, a coloring matter, a colorant, a stabilizer, and a flow agent. Examples of suitable coating materials include cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers, acrylic acid copolymers, methacrylic resins, zein, shellac, and polysaccharides, but are not limited thereto. Additionally, the coating materials may contain a typical carrier such as a plasticizer, a pigment, a colorant, a flow agent, a stabilizer, a pore former, and a surfactant. Optional pharmaceutically acceptable excipients include a diluent, a binder, a lubricant, a disintegrant, a colorant, a stabilizer, or a surfactant, but are not limited thereto.

As is known in the art, diluents are generally necessary to increase the volume of a solid dosage form, so that a particle size is provided for compression of tablets or formation of beads and granules. Suitable diluents include dicalcium phosphate dihydrate, calcium sulfate, lactose, sucrose, mannitol, sorbitol, cellulose, microcrystalline cellulose, kaolin, sodium chloride, dry starch, hydrolyzed starch, pregelatinized starch, silicone dioxide, titanium oxide, magnesium aluminum silicate or powdered sugar, but are not limited thereto. Binders are used to impart cohesive properties to a solid dosage formulation, and thus ensure that a tablet or bead or granule remains intact even after the composition of the dosage forms. Suitable binder materials include starch, pregelatinized starch, gelatin, sugars (including sucrose, glucose, dextrose, lactose and sorbitol), polyethylene glycol, waxes, natural and synthetic gums such as acacia, tragacanth, and sodium alginate, cellulose including hydroxypropylmethylcellulose, hydroxypropylcellulose, ethyl cellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone, but are not limited thereto. Furthermore, lubricants are used to facilitate tablet preparation. Examples of suitable lubricants include magnesium stearate, calcium stearate, stearic acid, glycerol behenate, polyethylene glycol, talc, and mineral oil, but are not limited thereto. Disintegrants are used to facilitate disintegration or breakup of the dosage form after administration, and generally include starch, sodium starch glycolate, sodium carboxymethyl starch, sodium carboxymethylcellulose, hydraxypropyl cellulose, pregelatinized starch, clays, cellulose, alginine, gums or cross linked polymers, such as cross-linked PVP, but are not limited thereto. In the art, it is known that stabilizers are used to inhibit or retard drug decomposition reactions which include, for example, oxidative reactions. Suitable stabilizers include antioxidants, butylated hydroxytoluene (BHT), ascorbic acid, and salts and esters thereof; vitamin E, tocopherol and salts thereof; sulfites such as sodium metabisulphite; cysteine and derivatives thereof; citric acid; propyl gallate; and butylated hydroxyanisole (BHA), but are not limited thereto.

In some embodiments, oral dosage formulations, such as capsules, tablets, solutions, and suspensions, may be formulated for controlled release. For example, one or more compounds and optional one or more additional active components may be formulated into nanoparticles, microparticles, and combinations thereof, and encapsulated in a soft or hard gelatin or non-gelatin capsule or dispersed in a dispersing medium to form an oral suspension or syrup. The particles may be formed of the drug and a controlled release polymer or matrix. Alternatively, the drug particles may be coated with one or more controlled release coating agents prior to incorporation into a finished dosage form.

In the practice of the methods of the disclosure, it may be required to administer a high initial dose of fibroblast at initiation of therapy, in order to generate a high plasma concentration. This may be achieved through parenteral administration of the compound. The preparation for parenteral administration may be prepared as an aqueous composition using a technology publicly known to the person skilled in the art. Generally, such compositions may be prepared as injectable formulations, for example, solutions or suspensions; solid forms such as micro or nanoparticles, suitable for use to prepare solutions or suspensions upon the addition of a reconstitution medium prior to injection; emulsions, such as water-in-oil (w/o) emulsions or oil-in-water (o/w) emulsions, and microemulsions thereof, liposomes, or emulsomes. The carrier may be a solvent or dispersion medium containing, for example, water, ethanol, one or more polyols (for example: glycerol, propylene glycol, and liquid polyethylene glycol), oils (for example: vegetable oils (for example: peanut oil, corn oil, sesame oil, and the like), and combinations thereof, but is not limited thereto. The suitable fluidity may be maintained by using a coating material, such as lecithin, by maintaining the required particle size in the case of dispersion, or by using a surfactant. In addition, it is possible to include an isotonic agent sugars or salts (for example: sodium chloride), but the isotonic agent is not limited thereto. Solutions or dispersions of the active compounds as a free acid, a free base or pharmaceutically acceptable salts may be prepared in water or another solvent or dispersing medium suitably mixed with one or more pharmaceutically acceptable excipients. Examples of the excipients include surfactants, dispersants, emulsifiers, pH modifying agents, and combinations thereof, but are not limited thereto. Suitable surfactants may be anionic, cationic, amphoteric or nonionic surface active agents. Suitable anionic surfactants include those containing carboxylate, sulfonate and sulfate ions, but are not limited thereto. Examples of anionic surfactants include sodium, potassium, and ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include quaternary ammonium compounds such as benzalkonium chloride, benzethonium chloride, cetrimonium bromide, stearyl dimethylbenzyl ammonium chloride, polyoxyethylene and coconut amine, but are not limited thereto. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-alanine, sodium N-lauryl-iminodipropionate, myristoamphoacetate, lauryl betaine, and lauryl sulfobetaine, but are not limited thereto.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Reduction of Systemic TNF-alpha by Valproic Acid Treated Fibroblasts Subsequent to PD-1 Antibody

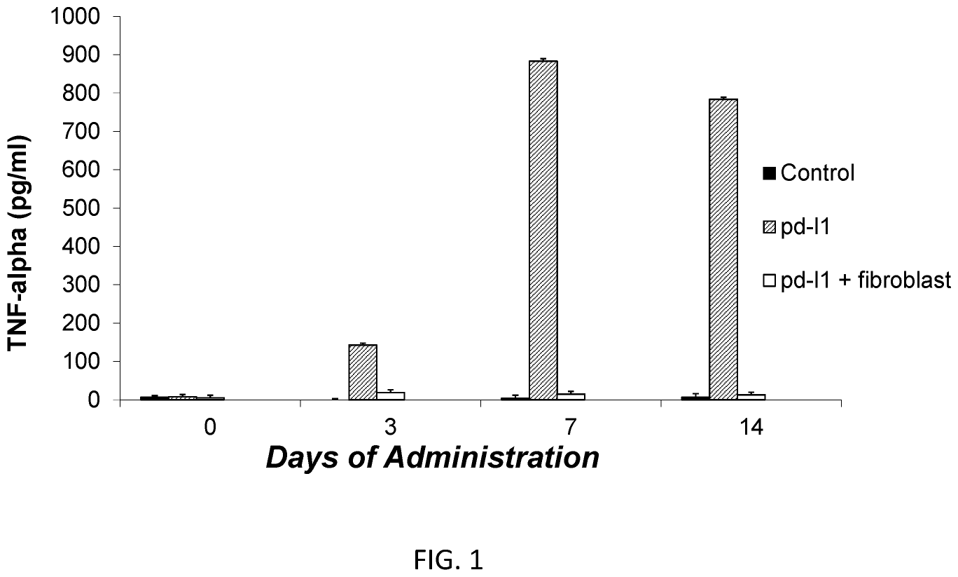

Balb/c mice were administered saline (control), antibody to PD-11 (PD-11) or antibody to PD-11 together with Balb/c fibroblasts (100,000 cells intraperitoneally) that had been cultured in valproic acid at 5 micrograms per milliliter for 24 hours. Cells and antibody were injected every second day. Serum TNF alpha was measured by ELISA (FIG. 1).

### Example 2

### Reduction of Lymphokine Activated Cell (LAK) Lethality by Administration of Fibroblasts

LAK cells were generated by culturing C57/BL6 splenocytes in 100 IU/ml interleukin-2, together with 10,000 anti-CD3, anti-CD28 beads per ml. Splenocytes were isolated by hypotoxic saline erythrocyte lysis followed by 2 washings in phosphate buffered saline (PBS). Cells were cultured in RPMI media with 10% fetal calf serum in a fully humidified atmosphere. Culture time was 96 hours, with cell viability assessed at the end of culture. Cells were assessed for cytotoxic activity against K562 target cells using the chromium⁵¹ release assay. One million cells per mouse were administered.

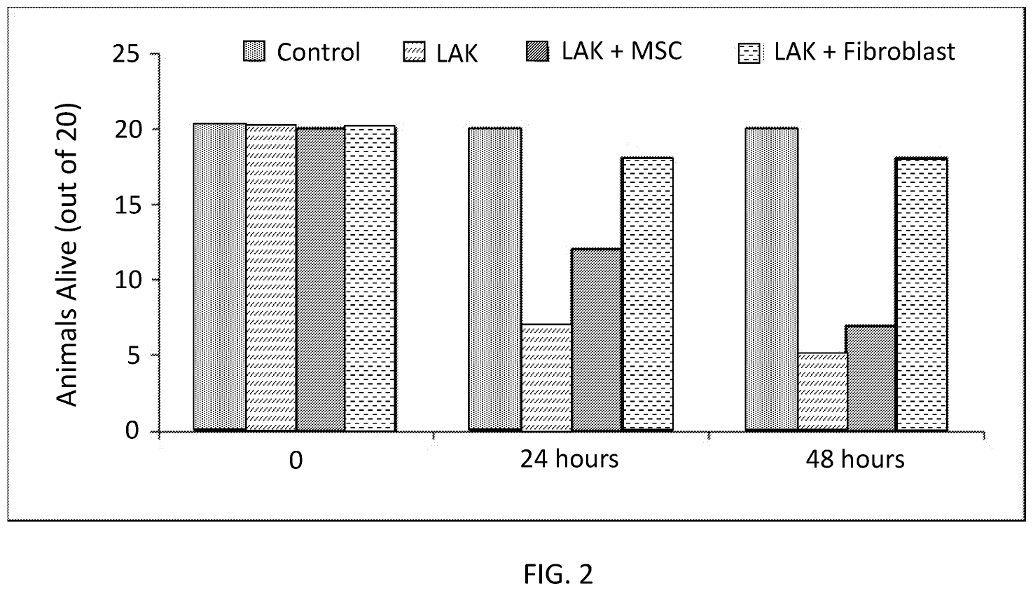

Fibroblasts or bone marrow MSCs were obtained from SpinalCyte (CybroCell dermal fibroblasts) or Allcells, respectively. Passage 3 cells were used and administered intravenously per mouse. As seen in FIG. 2, a substantial reduction in mortality was observed in the mice treated with fibroblasts as compared to MSC. Fibroblasts or MSC were injected 4 hours subsequent to administration of LAK cells.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of reducing toxicity of a therapy for an individual, comprising the step of providing an effective amount of fibroblasts and/or fibroblast-derived microvesicles to the individual with the therapy and/or before the therapy and/or after the therapy has been given to the individual.

2. The method of claim 1, wherein the therapy is selected from the group consisting of immunotherapy, radiation, drug toxicity, oxygen therapy, endocrine therapy, gene therapy, and a combination thereof.

3. The method of claim 1, wherein the therapy is for cancer, infectious disease, and/or autoimmunity.

4. The method of claim 2, wherein the immunotherapy comprises an antibody or functional fragment thereof.

5. The method of claim 4, wherein the antibody is a monoclonal antibody.

6. The method of claim 4, wherein the functional antibody fragment comprises a scFv.

7. The method of claim 1, wherein the therapy comprises cells.

8. The method of claim 2, wherein the immunotherapy comprises cells expressing one or more engineered T-cell receptors (TCR) or one or more chimeric antigen receptors (CAR) or both.

9. The method of claim 8, wherein the TCR or CAR targets a cancer antigen.

10. The method of claim 8, wherein the CAR comprises more than one costimulatory domain.

11. The method of claim 1, wherein the fibroblasts are dedifferentiated fibroblasts.

12. The method of claim 11, further comprising the step of dedifferentiating the fibroblasts.

13. The method of claim 11, wherein fibroblasts are or were dedifferentiated upon exposure to a sufficient amount of one or more dedifferentiating agents.

14. The method of claim 13, wherein the dedifferentiating agent is selected from the group consisting of one or more histone deacetylase (HDAC) inhibitors, one or more DNMT inhibitors, hypoxia, exposure to stem cells or fractions thereof, and a combination thereof.

15. The method of claim 14, wherein the HDAC inhibitor is valproic acid.

16. The method of claim 15, wherein the valproic acid is exposed to the fibroblasts at a concentration of 1-100 micrograms per milliliter for a period of 1-72 hours.

17. The method of claim 1, wherein the fibroblasts are derived from a tissue comprising regenerative properties.

18. The method of claim 17, wherein the tissue is umbilical cord, placenta, or a mixture thereof.

19. The method of claim 1, wherein the fibroblasts express one or more of CD105, CD117, and/or CD34.

20. The method of claim 1, wherein the fibroblasts comprise expression of rhodamine 123 efflux activity.

21. The method of claim 1, wherein the microvesicles comprise exosomes, apoptotic bodies, exosome-like particles, or a mixture thereof.

22. The method of claim 1, wherein the microvesicles are produced from culture of de-differentiated fibroblasts using anion exchange chromatography, high performance liquid chromatography (HPLC), or both.

23. The method of claim 1, wherein the microvesicles express one or more markers selected from the group consisting of a) CD63; b) CD9; c) MHC I; d) CD56; and e) a combination thereof.

24. The method of claim 1, wherein the fibroblasts and/or fibroblast-derived microvesicles are modified to reduce macrophage activation.

25. The method of claim 24, wherein the fibroblasts and/or fibroblast-derived microvesicles are comprised in polymer-augmented liposomes.

26. The method of claim 1, wherein the individual is provided an effective amount of activated protein C.

27. A method of treating or preventing cytokine release syndrome in an individual in need of a therapy and/or having received a therapy, comprising the step of providing an effective amount of fibroblasts and/or fibroblast-derived microvesicles to the individual with the therapy and/or before the therapy and/or after the therapy has been given to the individual.

28. The method of claim 27, wherein the cytokine release syndrome is from a therapy, an infectious disease, or a non-infectious disease.

29. The method of claim 28, wherein the non-infectious disease is graft-versus-host disease (GVHD), acute respiratory distress syndrome (ARDS), sepsis, sepsis, pancreatitis, burns, trauma, or Hemophagocytic lymphohistiocytosis.

30. The method of claim 28, wherein the infectious disease is Ebola, influenza, severe acute respiratory syndrome, malaria, or smallpox.

31. The method of claim 30, wherein the influenza is avian influenza.

32. The method of claim 30, wherein the influenza is Type A, Type B, or Type C influenza.

33. The method of claim 27, wherein the therapy is immunotherapy.

34. The method of claim 27, wherein the therapy comprises an antibody.

35. The method of claim 27, wherein the cytokine release syndrome is further defined as systemic inflammatory response syndrome, cytokine storm, cytokine cascade, or hypercytokinemia.

36. The method of claim 27, wherein the individual is further provided one or more corticosteroids, one or more biological therapies, and/or one or more anti-inflammatory agents.

37. The method of claim 36, wherein the biological therapy comprises one or more anti-IL6 therapies.

38. The method of claim 37, wherein the anti-IL6 therapy comprises an anti-IL6 antibody.

39. A method of reducing cytokine levels of one or more cytokines in an individual, comprising the step of providing an effective amount of fibroblasts and/or fibroblast-derived microvesicles to an individual in need of reduction of one or more cytokines.

40. The method of claim 39, wherein the individual has cytokine release syndrome.

41. The method of claim 40, wherein the cytokine release syndrome is further defined as systemic inflammatory response syndrome, cytokine storm, cytokine cascade or hypercytokinemia.

42. A method of treating cachexia in an individual, comprising the step of providing to the individual an effective amount of fibroblasts and/or fibroblast-derived microvesicles to an individual.

43. A method of enhancing efficacy of a therapy in an individual, comprising the step of providing to the individual an effective amount of fibroblasts and/or fibroblast-derived microvesicles to an individual.
